SEQUENCE LISTING (34 sequences)

SEQ ID NO: 1 - DNA, 363 bp, Mus musculus
  CDS (1)..(363)
gaggtgcatctggtggagtctgggggagacttagtgaggcctggagggtccctgaaactctcctgtgcagcctctggattcactttcggaagttatggcatgtcttgggttcgccagactgcagacaagaggctggagtgggtcgcaaccatttatagtggtggtttttacacctactatccagacagtgtgaggggacgattcaccatctccagagacaatgtcaaggaaatcgtgtatctgcaaatgagcagtctgaagtctgaggacacagccatgtattactgtgcaagacggacggtagtatctacggactatactttggactactggggtcaaggaacctcagtcatcgtctcctca

SEQ ID NO: 2 - protein, 121 aa, Mus musculus
EVHLVESGGDLVRPGGSLKLSCAASGFTFGSYGMSWVRQTADKRLEWVATIYSGGFYTYYPDSVRGRFTISRDNVKEIVYLQMSSLKSEDTAMYYCARRTVVSTDYTLDYWGQGTSVIVSS

SEQ ID NO: 3 - DNA, 318 bp, Mus musculus
  CDS (1)..(318)
gatatccagataacacagattacatcctccctggctgcctctctgggagacagagtcaccatcagttgccggccaagtcaggacatcagcaattttttaaactggtttcagcagaaaccagatggaactgttgaagtcctgatctgctacacattaagaatgcacttaggagtcccatcaacgttcagtggctgtgtgtctggaacatattatactctcaccagtagcaacctggaacaagaagatatagacacttcctttgccattaggattatacgcgtgctcacggtcggtgcagggaccacgctggagctgaaa

SEQ ID NO: 4 - protein, 106 aa, Mus musculus
DIQITQITSSLAASLGDRVTISCRPSQDISNFLNWFQQKPDGTVEVLICYTLRMHLGVPSTFSGCVSGTYYTLTSSNLEQEDIDTSFAIRIIRVLTVGAGTTLELK

SEQ ID NO: 5 - protein, 15 aa, Artificial Sequence
  Description of Artificial Sequence Synthetic linker
GGGGSGGGGSGGGGS

SEQ ID NO: 6 - protein, 14 aa, Artificial Sequence
  Description of Artificial Sequence Synthetic linker
GSTSGSGKSSEGKG

SEQ ID NO: 7 - protein, 18 aa, Artificial Sequence
  Description of Artificial Sequence Synthetic linker
GSTSGSGKSSEGSGSTKG

SEQ ID NO: 8 - protein, 12 aa, Artificial Sequence
  Description of Artificial Sequence Synthetic linker
GSTSGKPSEGKG

SEQ ID NO: 9 - protein, 18 aa, Artificial Sequence
  Description of Artificial Sequence Synthetic linker
GSTSGSGKPGSGEGSTKG

SEQ ID NO: 10 - DNA, 18 bp, Artificial Sequence
  Description of Artificial Sequence Synthetic primer
gcttgccgggtgggccac

SEQ ID NO: 11 - DNA, 18 bp, Artificial Sequence
  Description of Artificial Sequence Synthetic primer
acactgctggacagggat

SEQ ID NO: 12 - DNA, 28 bp, Artificial Sequence
  Description of Artificial Sequence Synthetic primer
ggatcccgggagtaccccttgaccaggc

SEQ ID NO: 13 - DNA, 18 bp, Artificial Sequence
  Description of Artificial Sequence Synthetic primer
gttgaagctcttgacaat

SEQ ID NO: 14 - DNA, 27 bp, Artificial Sequence
  Description of Artificial Sequence Synthetic primer
ggatcccgggtggatggtgggaagatg

SEQ ID NO: 15 - DNA, 36 bp, Artificial Sequence
  Description of Artificial Sequence Synthetic primer
  modified_base (24): inosine
  modified_base (25): inosine
  modified_base (29): inosine
  modified_base (30): inosine
  modified_base (34): inosine
  modified_base (35): inosine
ggccacgcgtcgactagtacgggnngggnngggnng

SEQ ID NO: 16 - DNA, 20 bp, Artificial Sequence
  Description of Artificial Sequence Synthetic primer
ggccacgcgtcgactagtac

SEQ ID NO: 17 - DNA, 35 bp, Artificial Sequence
  Description of Artificial Sequence Synthetic primer
actagtcgacatggtrtccwcasctcagttccttg

SEQ ID NO: 18 - DNA, 19 bp, Artificial Sequence
  Description of Artificial Sequence Synthetic primer
ggaaacagctatgaccatg

SEQ ID NO: 19 - DNA, 17 bp, Artificial Sequence
  Description of Artificial Sequence Synthetic primer
gtaaaacgacggccagt

SEQ ID NO: 20 - DNA, 58 bp, Artificial Sequence
  Description of Artificial Sequence Synthetic primer
attgttattactcgcggcccaaccggccatggccgaggtgcatctggtggagtctggg

SEQ ID NO: 21 - DNA, 59 bp, Artificial Sequence
  Description of Artificial Sequence Synthetic primer
ccgccggatccacctccgcctgaaccgcctccacctgaggagacgatgactgaggttcc

SEQ ID NO: 22 - DNA, 56 bp, Artificial Sequence
  Description of Artificial Sequence Synthetic primer
caggcggaggtggatccggcggtggcggatcggatatccagataacacagattaca

SEQ ID NO: 23 - DNA, 67 bp, Artificial Sequence
  Description of Artificial Sequence Synthetic primer
gctcaactttcttgtcgactttatcatcatcatctttataatctttcagctccagcgtggtccctgc

SEQ ID NO: 24 - DNA, 348 bp, Mus musculus
  CDS (1)..(348)
gatgtacaacttcaggagtcaggacctggcctcgtgaaaccttctgagtctctgtctctcacctgttctgtcactggctactccatcaccagtggttattactggaattggatccggcaatttccaggaaacaaactggattggatgggccatataagttacgacggtaacaataactacaacccatctctcaaaaatcgaatctccatcactcgtgacacatctaagaaccagtttttcctgaagttgaattctgtgactactgaggacacagatacatattactgttctatgatcctctatggtatggactactggggtcagggaacctcagtcaccgtctcctca

SEQ ID NO: 25 - protein, 116 aa, Mus musculus
DVQLQESGPGLVKPSESLSLTCSVTGYSITSGYYWNWIRQFPGNKLDWMGHISYDGNNNYNPSLKNRISITRDTSKNQFFLKLNSVTTEDTDTYYCSMILYGMDYWGQGTSVTVSS

SEQ ID NO: 26 - DNA, 324 bp, Mus musculus
  CDS (1)..(324)
cagattgttctcacccagtctccagcaatcatgtctgcatctctaggggaacgggtcaccatgacctgcactgccagctcaagtgtaagttccagttacttgcactggtaccagcagaagccaggatcctcccccaaactctgcatttatagcacatccaacctggcttctggagtcccaactcgcttcagtggcagtgggtctgggacctcttactctctcacaataagcagcatggaggctgaagatgctgccacttattactgccaccagtatcatcgttccccacccacgttcggctcggggacaaagttggaaataaaa

SEQ ID NO: 27 - protein, 108 aa, Mus musculus
QIVLTQSPAIMSASLGERVTMTCTASSSVSSSYLHWYQQKPGSSPKLCIYSTSNLASGVPTRFSGSGSGTSYSLTISSMEAEDAATYYCHQYHRSPPTFGSGTKLEIK

SEQ ID NO: 28 - DNA, 18 bp, Artificial Sequence
  Description of Artificial Sequence Synthetic primer
gctggccgggtgggcaac

SEQ ID NO: 29 - DNA, 40 bp, Artificial Sequence
  Description of Artificial Sequence Synthetic primer
actagtcgacatggatttwcaggtgcagattwtcagcttc

SEQ ID NO: 30 - DNA, 60 bp, Artificial Sequence
  Description of Artificial Sequence Synthetic primer
attgttattactcgcggcccaaccggccatggccgatgtacaacttcaggagtcaggacc

SEQ ID NO: 31 - DNA, 61 bp, Artificial Sequence
  Description of Artificial Sequence Synthetic primer
ccgccggatccacctccgcctgaaccgcctccacctgaggagacggtgactgaggttccct

SEQ ID NO: 32 - DNA, 55 bp, Artificial Sequence
  Description of Artificial Sequence Synthetic primer
caggcggaggtggatccggcggtggcggatcgcagattgttctcacccagtctcc

SEQ ID NO: 33 - DNA, 66 bp, Artificial Sequence
  Description of Artificial Sequence Synthetic primer
gctcaactttcttgtcgactttatcatcatcatctttataatcttttatttccaactttgtccccg

SEQ ID NO: 34 - protein, 5 aa, Artificial Sequence
  Description of Artificial Sequence Synthetic linker
GGGGS

(19) United States
(12) Patent Application Publication
(10) Pub. No.: US 2007/0072234 A1
(43) Pub. Date: Mar. 29, 2007

(54) Protein capable of binding plasticizer

(75) Inventors: Norihiro Kobayashi, Kobe (JP); Yasuhiro Goda, Osaka (JP); Masato Hirobe, Osaka (JP)

Correspondence Address: EDWARDS & ANGELL, LLP, P.O. BOX 55874, BOSTON, MA, 02205, US

(73) Assignee: Japan EnviroChemicals, Ltd., Osaka (JP)

(21) Appl. No.: 10/553,305

(22) PCT Filed: Apr. 13, 2004
(86) PCT No.: PCT/JP04/05250

§ 371 Date: Sep. 26, 2006

(30) Foreign Application Priority Data
Apr. 15, 2003 (JP) 2003-110877

(51) Int. Cl. G01N 33/53; C07K 14/47; C07K 16/18

(52) U.S. Cl. 435/7.1; 530/350; 530/388.1

(57) ABSTRACT

The present invention provides a protein having a binding capacity to a plasticizer, which has been conferred with useful properties for measuring, quantifying, or concentrating a plasticizer, such as high sensitivity, low cross-reactivity, high tolerance for interferents, and high tolerance for solvents. Specifically, the present invention provides a modified protein having various properties, such as affinity for antigenic plasticizers, antigen binding capacity, cross-reactivity, tolerance for antigen-antibody reaction interferents, tolerance for enzymatic color developing reaction interferents, and tolerance for solvents, improved by gene recombination technology.

### TECHNICAL FIELD

The present invention relates to an anti-plasticizer antibody, a gene for the antibody, a method of producing a protein having a binding capacity to a plasticizer, a method of measuring or quantifying a plasticizer, a method of concentrating a plasticizer, and the like.

### BACKGROUND ART

In recent years, environmental pollution with environmental pollutants, such as plasticizers, which are present in the environment, for example, in river water or sewage, has been problematic. It is therefore necessary to measure and analyze environmental pollutants and degradation products thereof in the environment, and to make use of the results for environmental conservation. Some excellent methods of such measurements and analyses are known (see, for example, WO99/43799 and JP-A-2001-41958).

### DISCLOSURE OF THE INVENTION

The present invention is directed to prepare and utilize a protein having a binding capacity to a plasticizer, obtained by conferring useful properties for measuring, quantifying or concentrating plasticizers, such as good sensitivity, low cross-reactivity, high tolerance for interferents, and high tolerance for solvents, to a modified protein obtained by identifying the gene for an antibody against a plasticizer, and improving, by modification technology for gene manipulation, various properties of the original antibody, such as antigen affinity, antigen binding capacity, cross-reactivity, tolerance for antigen-antibody reaction inteferents, tolerance for enzymatic color developing reaction interferents, and tolerance for solvents.

As examples of the plasticizer, plasticizers (PP) represented by formula (1): 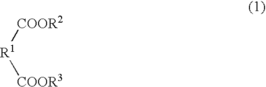
wherein R¹ represents o-phenylene or tetramethylene, and R² and R³ are the same or different and each represents H, a linear or branched (including sec-, tert-, and iso-) alkyl having 1 to 20 carbon atoms, a benzyl that may be substituted, or a cyclohexyl that may be substituted] [e.g., BBP (butylbenzyl phthalate), DBP (dibutyl phthalate), DCHP (dicyclohexyl phthalate), DEP (diethyl phthalate), DEHP (di(2-ethylhexyl) phthalate), DEHA (diethylhexyl adipate), DHP (dihexyl phthalate), DPP (di-n-pentyl phthalate), DPrP (dipropyl phthalate), DMP (dimethyl phthalate), DnOP (di-normal-octyl phthalate), DINP (diisononyl phthalate), DNP (dinonyl phthalate), DIDP (diisodecyl phthalate), DOA (dioctyl adipate), DINA (diisononyl adipate) and the like] can be mentioned.

As examples of the “linear or branched alkyl having 1 to 20 carbon atoms”, methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl, heptyl, octyl, 2-ethylhexyl, nonyl, isononyl, decyl, isodecyl and the like can be mentioned. As the “linear or branched alkyl” in the above-described “linear or branched alkyl having 1 to 20 carbon atoms”, an alkyl having 1 to 12 carbon atoms is preferred, with greater preference given to an alkyl having 6 to 10 carbon atoms.

In another aspect, the “linear or branched alkyl having 1 to 20 carbon atoms” can be an alkyl having 1 to 20 carbon atoms that may be substituted. As examples of the “alkyl” in the above-described “alkyl having 1 to 20 carbon atoms that may be substituted”, the same as the examples of the “alkyl” in the above-described “linear or branched alkyl having 1 to 20 carbon atoms” can be mentioned, and an alkyl having 1 to 12 carbon atoms is preferred, with greater preference given to an alkyl having 4 to 8 carbon atoms.

As examples of the substituent for the “alkyl having 1 to 20 carbon atoms that may be substituted”, the “cyclohexyl that may be substituted” and the “benzyl that may be substituted”, an alkyl having 1 to 8 carbon atoms (for example, methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl and the like), an alkenyl having 2 to 8 carbon atoms (for example, etenyl, 1-propenyl, 2-propenyl, 1-methylethenyl, 1-butenyl, 2-butenyl, 3-butenyl, 1-methyl-1-propenyl, 1-methyl-2-propenyl, 2-methyl-1-propenyl and the like), an alkynyl having 2 to 8 carbon atoms (for example, ethynyl, 1-propynyl, 2-propynyl, 1-butynyl, 2-butynyl, 3-butynyl, 1-methyl-2-propynyl and the like) and the like can be mentioned.

As the above-described “alkyl having 1 to 8 carbon atoms”, an alkyl having 1 to 6 carbon atoms is preferred, with greater preference given to an alkyl having 1 to 4 carbon atoms. As the above-described “alkenyl having 2 to 8 carbon atoms”, an alkenyl having 2 to 6 carbon atoms is preferred, with greater preference given to an alkenyl having 2 to 4 carbon atoms. As the above-described “alkynyl-having 2 to 8 carbon atoms”, an alkynyl having 2 to 6 carbon atoms is preferred, with greater preference given to an alkynyl having 2 to 4 carbon atoms. Although the number of substituents for the “alkyl having 1 to 20 carbon atoms that may be substituted”, the “cyclohexyl that may be substituted”, and the “benzyl that may be substituted” is not subject to limitation, it can be, for example, 1 to 3, preferably 1 to 2, more preferably 1.

As other examples of the plasticizer, DOZ (dioctyl azelate), ESBO (epoxidized soybean oil), TOTM (trioctyl trimellitate), DBS (dibutyl sebacate), DOS (dioctyl sebacate), TCP (tricresyl phosphate), ATBC (acetyltributyl citrate) and the like can be mentioned.

The present inventors have conducted extensive studies of obtaining a protein having a binding capacity to an anti-plasticizer, which is conferred with useful properties such as being capable of highly sensitive measurements, by improving affinity, and found it possible to prepare a transformant containing its gene or an modified gene, and to efficiently produce the protein having a binding capacity to the plasticizer, and conducted further studies to complete the present invention.

Accordingly, the present invention provides:

(1) a protein of the following (a) or (b), or a salt thereof:

(a) a protein having the amino acid sequence shown by SEQ ID NO:2, the amino acid sequence shown by SEQ ID NO:25, or an amino acid sequence substantially the same as these;

(b) a protein having the amino acid sequence shown by SEQ ID NO:4, the amino acid sequence shown by SEQ ID NO:27, or an amino acid sequence substantially the same as these,

(2) a protein of any of the following (a1) to (a4) and (b1) to (b4), or a salt thereof:

(a1) a protein having an amino acid sequence having one or two or more amino acids deleted, substituted or added in the amino acid sequence shown by SEQ ID NO:2, which binds to a plasticizer when forming a complex with the amino acid sequence shown by SEQ ID NO:4 or SEQ ID NO:27;

(a2) a protein having an amino acid sequence having one or two or more amino acids deleted, substituted or added in the amino acid sequence shown by SEQ ID NO:2, which binds to a plasticizer hen forming a complex with a protein having an amino acid sequence having one or two or more amino acids deleted, substituted or added in the amino acid sequence shown by SEQ ID NO:4 or SEQ ID NO:27;

(a3) a protein having an amino acid sequence having one or two or more amino acids deleted, substituted or added in the amino acid sequence shown by SEQ ID NO:25, which binds to a plasticizer when forming a complex with the amino acid sequence shown by SEQ ID NO:4 or SEQ ID NO:27;

(a4) a protein having an amino acid sequence having one or two or more amino acids deleted, substituted or added in the amino acid sequence shown by SEQ ID NO:25, which binds to a plasticizer when forming a complex with a protein having an amino acid sequence having one or two or more amino acids deleted, substituted or added in the amino acid sequence shown by SEQ ID NO:4 or SEQ ID NO:27;

(b1) a protein having an amino acid sequence having one or two or more amino acids deleted, substituted or added in the amino acid sequence shown by SEQ ID NO:4, which binds to a plasticizer when forming a complex with a protein having the amino acid sequence shown by SEQ ID NO:2 or SEQ ID NO:25;

(b2) a protein having an amino acid sequence having one or two or more amino acids deleted, substituted or added in the amino acid sequence shown by SEQ ID NO:4, which binds to a plasticizer when forming a complex with a protein having an amino acid sequence having one or two or more amino acids deleted, substituted or added in the amino acid sequence shown by SEQ ID NO:2 or SEQ ID NO:25:

(b3) a protein having an amino acid sequence having one or two or more amino acids deleted, substituted or added in the amino acid sequence shown by SEQ ID NO:27, which binds to a plasticizer when forming a complex with a protein having the amino acid sequence shown by SEQ ID NO:2 or SEQ ID NO:25:

(b4) a protein having an amino acid sequence having one or two or more amino acids deleted, substituted or added in the amino acid sequence shown by SEQ ID NO:27, which binds to a plasticizer when forming a complex with a protein having an amino acid sequence having one or two or more amino acids deleted, substituted or added in the amino acid sequence shown by SEQ ID NO:2 or SEQ ID NO:25,

(3) a protein of the following (a) or (b), or a salt thereof:

(a) a protein having the amino acid sequence shown by SEQ ID NO:2 or an amino acid sequence substantially the same as these;

(b) a protein having the amino acid sequence shown by SEQ ID NO:4 or an amino acid sequence substantially the same as these,

(4) the protein of (2) or (3) above, wherein the plasticizer is a plasticizer represented by formula (1): 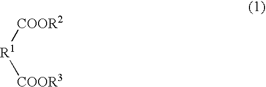
wherein R¹ represents o-phenylene, and R² and R³ are the same or different and each represents H, a linear or branched alkyl having 1 to 20 carbon atoms, a benzyl that may be substituted, or a cyclohexyl that may be substituted,

(5) a method of genetically recombining a protein of any one of (1) to (4) and (6) above,

(6) a protein obtained by the method of (5) above, or a salt thereof,

(7) a partial peptide of a protein of any one of (1) to (4) and (6) above, or a salt thereof,

(8) a polynucleotide encoding a protein of any one of (1) to (4) and (6) above, or a partial peptide thereof,

(9) a recombinant vector harboring the polynucleotide (8) above,

(10) a transformant transformed with the recombinant vector (9) above,

(11) a method of producing a protein of any one of (1) to (4) and (6) above, a partial peptide thereof, or a salt thereof, which comprises producing a protein of any one of (1) to (4) and

(6) above, a partial peptide thereof, or a salt thereof and harvesting the same,

(12) a complex wherein the following (a) and (b) are linked:

(a) a protein having the amino acid sequence shown by SEQ ID NO:2, the amino acid sequence shown by SEQ ID NO:25, or an amino acid sequence substantially the same as these;

(b) a protein having the amino acid sequence shown by SEQ ID NO:4, the amino acid sequence shown by SEQ ID NO:27, or an amino acid sequence substantially the same as these,

(13) a method of identifying a plasticizer that binds to the complex (12) above, which comprises using the complex,

(14) a method of measuring or quantifying a plasticizer, which comprises using the complex (12) above,

(15) a kit for measuring or quantifying a plasticizer, which comprises the complex (12) above,

(16) a method of concentrating a plasticizer, which comprises using the complex (12) above,

(17) a kit for concentrating a plasticizer, which comprises the complex (12) above,

and the like.

### BRIEF DESCRIPTION OF THE DRAWINGS

FIG. 1 shows the base sequence and amino acid sequence of the heavy chain of an anti-plasticizer antibody (DH-150).

FIG. 2 shows the base sequence and amino acid sequence of the light chain of an anti-plasticizer antibody (DH-150).

FIG. 3 shows an agarose gel electrophoresis of a single chain antibody gene having the heavy and light chains of an anti-plasticizer antibody (DH-150).

FIG. 4 shows the base sequence and amino acid sequence of the heavy chain of an anti-plasticizer antibody (DF-34).

FIG. 5 shows the base sequence and amino acid sequence of the light chain of an anti-plasticizer antibody (DH-34).

FIG. 6 shows a comparison of the base sequences and amino acid sequences of the heavy chains of anti-plasticizer antibodies (DF-150, DF-34).

FIG. 7 shows a comparison of the base sequences and amino acid sequences of the light chains of anti-plasticizer antibodies (DF-150, DF-34).

### DETAILED DESCRIPTION OF THE INVENTION

The present invention provides a protein having (or consisting of) the amino acid sequence shown by SEQ ID NO:2, the amino acid sequence shown by SEQ ID NO:25, the amino acid sequence shown by SEQ ID NO:4, the amino acid sequence shown by SEQ ID NO:27, or an amino acid sequence substantially the same as these.

In an embodiment of the invention, the protein having an amino acid sequence substantially the same as the amino acid sequence shown by SEQ ID NO:2 can be the protein (a1) or (a2) above, (a5) a protein having (or consisting of) an amino acid sequence wherein an amino acid sequence corresponding to 1 or more particular regions in the amino acid sequence shown by SEQ ID NO:2 has been exchanged with an amino acid sequence corresponding to 1 or more particular regions of the same kind contained in the amino acid sequence of the heavy chain variable region of another antibody against a plasticizer (for example, the amino acid sequence shown by SEQ ID NO:25), or a protein having (or consisting of) an amino acid sequence substantially the same as these. As examples of the protein having an amino acid sequence substantially the same as the amino acid sequence of the protein (a5), a protein having an amino acid sequence having one or two or more amino acids deleted, substituted or added in the amino acid sequence of the protein (a5), which binds to a plasticizer when forming a complex with the protein (b), can be mentioned.

In another embodiment, the protein having an amino acid sequence substantially the same as the amino acid sequence shown by SEQ ID NO:25 can be the protein (a3) or (a4) above, (a6) a protein having (or consisting of) an amino acid sequence wherein an amino acid sequence corresponding to 1 or more particular 30 regions in the amino acid sequence shown by SEQ ID NO:25 has been exchanged with an amino acid sequence corresponding to 1 or more particular regions of the same kind contained in the amino acid sequence of the heavy chain variable region of another antibody against a plasticizer (for example, the amino acid sequence shown by SEQ ID NO:2), or a protein having (or consisting of) an amino acid sequence substantially the same as these. As examples of the protein having an amino acid sequence substantially the same as the amino acid sequence of the protein (a6), a protein having an amino acid sequence having one or two or more amino acids deleted, substituted or added in the amino acid sequence of the protein (a6), which binds to a plasticizer when forming a complex with the protein (b), can be mentioned.

As the particular region in the (a5) and (a6) above, complementarity determining region 1, complementarity determining region 2, complementarity determining region 3 (hereinafter to be abbreviated as CDR1, CDR2, CDR3 as necessary), framework region 1, framework region 2, framework region 3, framework region 4 (hereinafter to be abbreviated as FR1, FR2, FR3, FR4 as necessary) can be mentioned. In the (a5) and (a6) above, the amino acid sequence to be the object of change is preferably an amino acid sequence of the same kinds of particular regions. In addition, while the number of the particular regions to be changed is not particularly limited as long as it is not less than 1, it is, for example, 1 to 3, preferably 1 or 2, more preferably 1. The amino acid sequences can be exchanged by a method known per se. Specifically, a primer wherein a part corresponding to the regions to be changed is linked to a primer corresponding to both the N, C terminals of each region is designed, and using this primer, a fragment is amplified by PCR, and a PCR is performed with the exchanged combination.

The regions corresponding to CDR1, CDR2, CDR3, FR1, FR2, FR3, and FR4 in the amino acid sequence shown by SEQ ID NO:2 are specifically as follows:

(i) CDR1 (the 31st to 35th amino acid residues in the amino acid sequence shown by SEQ ID NO:2);

(ii) CDR2 (the 50th to 66th amino acid residues in the amino acid sequence shown by SEQ ID NO:2);

(iii) CDR3 (the 99th to 110th amino acid residues in the amino acid sequence shown by SEQ ID NO:2);

(iv) FR1 (the 1st to 30th amino acid residues in the amino acid sequence shown by SEQ ID NO:2);

(v) FR2 (the 36th to 49th amino acid residues in the amino acid sequence shown by SEQ ID NO:2);

(vi) FR3 (the 67th to 98th amino acid residues in the amino acid sequence shown by SEQ ID NO:2);

(vii) FR4 (the 111th to 121st amino acid residues in the amino acid sequence shown by SEQ ID NO:2).

The regions corresponding to CDR1, CDR2, CDR3, FR1, FR2, FR3, and FR4 in the amino acid sequence shown by SEQ ID NO:25 are specifically as follows:

(i) CDR1 (the 31st to 36th amino acid residues in the amino acid sequence shown by SEQ ID NO:25);

(ii) CDR2 (the 51st to 66th amino acid residues in the amino acid sequence shown by SEQ ID NO:25);

(iii) CDR3 (the 99th to 105th amino acid residues in the amino acid sequence shown by SEQ ID NO:25);

(iv) FR1 (the 1st to 30th amino acid residues in the amino acid sequence shown by SEQ ID NO:25);

(v) FR2 (the 37th to 50th amino acid residues in the amino acid sequence shown by SEQ ID NO:25);

(vi) FR3 (the 67th to 98th amino acid residues in the amino acid sequence shown by SEQ ID NO:25);

(vii) FR4 (the 106th to 116th amino acid residues in the amino acid sequence shown by SEQ ID NO:25).

In another embodiment, the protein (a) above can be, for example, a protein having the amino acid sequence shown by SEQ ID NO:2 or SEQ ID NO:25, or an amino acid sequence having significant homology to the amino acid sequence of the protein (a5) or (a6) above, which binds to a plasticizer when forming a complex with a protein having an amino acid sequence having significant homology to the amino acid sequence of the protein (b).

In an embodiment, the protein having an amino acid sequence substantially the same as the amino acid sequence shown by SEQ ID NO:4 can be the protein (b1) or (b2) above, (b5) a protein having (or consisting of) an amino acid sequence wherein an amino acid sequence corresponding to 1 or more particular regions in the amino acid sequence shown by SEQ ID NO:4 has been exchanged with an amino acid sequence corresponding to 1 or more particular regions of the same kind contained in the amino acid sequence of the light chain variable region of another antibody against a plasticizer (for example, the amino acid sequence shown by SEQ ID NO:27), or a protein having (or consisting of) an amino acid sequence substantially the same as these. As examples of the protein having an amino acid sequence substantially the same as the amino acid sequence of the protein (b5), a protein having an amino acid sequence having one or two or more amino acids deleted, substituted or added in the amino acid sequence of the protein (b5), which binds to a plasticizer when forming a complex with the protein (a), can be mentioned.

In another embodiment, the protein having an amino acid sequence substantially the same as the amino acid sequence shown by SEQ ID NO:27 can be the protein (b3) or (b4) above, (b6) a protein having (or consisting of) an amino acid sequence wherein an amino acid sequence corresponding to 1 or more particular regions in the amino acid sequence shown by SEQ ID NO:27 has been exchanged with an amino acid sequence corresponding to 1 or more particular regions of the same kind contained in the amino acid sequence of the light chain variable region of another antibody against a plasticizer (for example, the amino acid sequence shown by SEQ ID NO:4), or a protein having (or consisting of) an amino acid sequence substantially the same as these. As examples of the protein having an amino acid sequence substantially the same as the amino acid sequence of the protein (b6), a protein having an amino acid sequence having one or two or more amino acids deleted, substituted or added in the amino acid sequence of the protein (b6), which binds to a plasticizer when forming a complex with the protein (a), can be mentioned. As the particular region in the (b5) and (b6) above, CDR1, CDR2, CDR3, FR1, FR2, FR3, and FR4 can be mentioned. In the (b5) and (b6) above, the amino acid sequence to be exchanged is preferably an amino acid sequence of the same kind of particular regions. Although the number of particular regions exchanged is not subject to limitation, as long as it is not less than 1, it is, for example, 1 to 3, preferably 1 to 2, and more preferably 1. Amino acid sequences can be exchanged by a method known per se. Specifically, a primer wherein a portion corresponding to the regions to be exchanged is linked to a primer corresponding to both the N and C terminals of each region is designed, and using this primer, a fragment is amplified by PCR, and subsequently a PCR is performed with the exchanged combination.

The regions corresponding to CDR1, CDR2, CDR3, FR1, FR2, FR3, and FR4 in the amino acid sequence shown by SEQ ID NO:4 are specifically as follows:

(i) CDR1 (the 24th to 34th amino acid residues in the amino acid sequence shown by SEQ ID NO:4);

(ii) CDR2 (the 50th to 56th amino acid residues in the amino acid sequence shown by SEQ ID NO:4);

(iii) CDR3 (the 89th to 96th amino acid residues in the amino acid sequence shown by SEQ ID NO:4);

(iv) FR1 (the 1st to 23rd amino acid residues in the amino acid sequence shown by SEQ ID NO:4);

(v) FR2 (the 35th to 49th amino acid residues in the amino acid sequence shown by SEQ ID NO:4);

(vi) FR3 (the 57th to 88th amino acid residues in the amino acid sequence shown by SEQ ID NO:4);

(vii) FR4 (the 97th to 106th amino acid residues in the amino acid sequence shown by SEQ ID NO:4).

The regions corresponding to CDR1, CDR2, CDR3, FR1, FR2, R3, and FR4 in the amino acid sequence shown by SEQ ID NO:27 are specifically as follows:

(i) CDR1 (the 24th to 35th amino acid residues in the amino acid sequence shown by SEQ ID NO:27);

(ii) CDR2 (the 51st to 57th amino acid residues in the amino acid sequence shown by SEQ ID NO:27);

(iii) CDR3 (the 90th to 98th amino acid residues in the amino acid sequence shown by SEQ ID NO:27);

(iv) FR1 (the 1st to 23rd amino acid residues in the amino acid sequence shown by SEQ ID NO:27);

(v) FR2 (the 36th to 50th amino acid residues in the amino acid sequence shown by SEQ ID NO:27);

(vi) FR3 (the 58th to 89th amino acid residues in the amino acid sequence shown by SEQ ID NO:27);

(vii) FR4 (the 99th to 108th amino acid residues in the amino acid sequence shown by SEQ ID NO:27).

In another embodiment, the protein (b) above can be, for example, a protein having the amino acid sequence shown by SEQ ID NO:4 or SEQ ID NO:25, or an amino acid sequence having significant homology to the amino acid sequence of the protein (b5) or (b6) above, which binds to a plasticizer when forming a complex with a protein having an amino acid sequence having significant homology to the amino acid sequence of the protein (a).

In the present invention, the number of amino acids deleted, substituted or added in the amino acid sequence shown by any SEQ ID NO: X is not subject to limitation, as long as it is 1 or 2 or more, and can, for example, be 1 to 80, preferably about 1 to 20, more preferably about 1 to 9, still more preferably 1 to 5, and most preferably several (1 or 2).

In the present invention, amino acid substitution is not subject to limitation, as long as a particular amino acid is substituted with an optionally chosen amino acid, and can, for example, be conservative amino acid substitution or non-conservative amino acid substitution. “Conservative amino acid substitution” refers to substituting a particular amino acid with an amino acid having a side chain of similar nature. Specifically, in conservative amino acid substitution, a particular amino acid is substituted with another amino acid belonging to the same group. On the other hand, “non-conservative amino acid substitution” refers to substituting a particular amino acid with another amino acid having a side chain of different nature. Specifically, in non-conservative amino acid substitution, a particular amino acid is substituted with another amino acid belonging to a different group. Groups of amino acids having a side chain of similar nature are known in the art. As such groups of amino acids, for example, amino acids having a basic (i.e., positively charged) side chain (e.g., lysine, arginine, histidine), amino acids having an acidic (i.e., negatively charged) side chain (e.g., aspartic acid, glutamic acid), and amino acids having a neutral (i.e., uncharged) side chain (e.g., glycine, asparagine, glutamine, serine, threonine, tyrosine, cysteine, alanine, valine, leucine, isoleucine, proline, phenylalanine, methionine, tryptophan). Amino acids having a neutral side chain can further be classified into amino acids having a polar side chain (e.g., glycine, asparagine, glutamine, serine, threonine, tyrosine, cysteine) and amino acids having a non-polar side chain (e.g., alanine, valine, leucine, isoleucine, proline, phenylalanine, methionine, tryptophan). Other groups can include, for example, amino acids having an aromatic side chain (e.g., phenylalanine, tryptophan, histidine), amino acids having a side chain containing a hydroxyl group (alcoholic hydroxyl group, phenolic hydroxyl group) (e.g., serine, threonine, tyrosine), and the like.

As amino acid sequences having significant homology to the amino acid sequence shown by any SEQ ID NO: X, for example, amino acid sequences having homology to the amino acid sequence shown by any SEQ ID NO: X of about 40% or more, preferably about 0% or more, more preferably about 80% or more, still more preferably about 90% or more, and most preferably about 95% or more can be mentioned.

Degree of homology (%) can be determined by a method known per se. For example, degree of homology (%) can be determined using the Gap program employing the algorithm of Smith and Waterman (Adv. Appl. Math., 1981, 2, 482-489) (Wisconsin Sequence Analysis Package, Version 8 for Unix (trade mark), Genetics Computer Group, University Research Park, Madison Wis.) in default settings. The BLAST program employing the algorithm of Karlin and Altschul (Proc. Natl. Acad. Sci. USA, 1990, 87:2264-2268, Proc. Natl. Acad. Sci. USA, 1993, 90:5873-5877) can also be used. For example, when comparing protein homologies, degree of homology (%) can be determined using the XBLAST program in default settings. Furthermore, the ALIGN program (version 2.0) (a portion of GCG sequence alignment software package) employing the algorithm of Myers and Miller (CABIOS, 1988, 4:11-17) can also be used. As settings for comparing amino acid sequences using the ALIGN program, for example, “PAM120 weight residue table, gap length penalty=12, gap penalty=4” can be mentioned. These programs can also be used, in similar manners, for determining degree of homology (%) of base sequences.

“Binding to a plasticizer when forming a complex” means that the complex has reactivity to the plasticizer. As examples of the plasticizer, those described above can be mentioned. Whether or not the complex has a binding capacity to the plasticizer can be determined by a method known per se or a method based thereon. The complex of the present invention may have binding capacity to any one of the above-described plasticizers.

By introducing a deletion, substitution or addition of one or more amino acids to a protein having the amino acid sequence shown by SEQ ID NO:2 or SEQ ID NO:25, SEQ ID NO:4 or SEQ ID NO:27, and the proteins (a5), (a6), (b5), and (b6), a protein having an altered binding capacity or cross-reactivity to a plasticizer can be obtained. The region in which one or more amino acids are deleted, substituted or added can be any one or more regions optionally selected from the group consisting of CDR1, CDR2, CDR3, FR1, FR2, FR3, and FR4.

The partial peptide of the present invention may be any peptide, as long as it is a peptide that constitutes a portion of the protein (a) or (b) above; there may be used, for example, a peptide consisting of at least 6 or more, preferably at least 8 or more, more preferably at least 10 or more, still more preferably at least 12 or more, and most preferably 15 or more consecutive amino acids in the amino acid sequence of the protein (a) or (b) above. As the partial peptide of the present invention, there can also be used a partial peptide having (or consisting of) an amino acid sequence corresponding to the CDR1, CDR2, CDR3, FR1, FR2, FR3, or FR4 of the protein (a) or protein (b) above.

As salts of the protein of the present invention or partial peptide thereof, there may be used known salts per se, for example, acid adduct salts. These acid adduct salts include, for example, salts with inorganic acids (e.g., hydrochloric acid, phosphoric acid, hydrobromic acid, sulfuric acid) or salts with organic acids (e.g., acetic acid, formic acid, propionic acid, fumaric acid, maleic acid, succinic acid, tartaric acid, citric acid, malic acid, oxalic acid, benzoic acid, methanesulfonic acid and benzenesulfonic acid).

In the present invention, the “complex” may be any one, as long as the protein (a) above and the protein (b) above are linked together, and is exemplified by a complex wherein the protein (a) above and the protein (b) above are covalently bound in the presence or absence of a linker. This complex can also be used in the form of a salt (preferably an acid adduct salt) as with the aforementioned protein and partial peptide.

As the linker for fusing the protein (a) above and the protein (b) above, there may be used without limitation any linker known in the art; such linkers include, for example, peptides of repeated sequences of GGGGS (SEQ ID NO:34) (such as GGGGSGGGGSGGGGS (SEQ ID NO:5)), GSTSGSGKSSEGKG (SEQ ID NO:6), GSTSGSGKSSEGSGSTKG (SEQ ID NO:7), GSTSGKPSEGKG (SEQ ID NO:8), GSTSGSGKPGSGEGSTKG (SEQ ID NO:9) etc. [see, for example, Production of single-chain Fv monomers and multimers, D. Filpula, J. McGuire, and M. Whitlow. In “Antibody Engineering” edited by J. McCafferty, H. R. Hoogenboon, and D. J. Chiswell. pp. 253-268, IRL PRESS (1996)]. A complex wherein the protein (a) above and the protein (b) above are covalently bound in the presence or absence of a linker can, for example, be obtained by separately preparing the protein (a) above and the protein (b) above, and subsequently covalently binding these proteins via a linker or by a direct covalent linkage. However, this method is painstaking because it requires a further step to link the protein (a) above and the protein (b) above after their preparation, so as to obtain their complex. Another drawback resides in that a plurality of complexes with different covalent bond sites can result in, so that a single complex that is preferred from the viewpoint of reproducibility etc. is difficult to be prepared. Therefore, the complex of the present invention is, for example, preferably a single-chain antibody wherein the protein (a) above and the protein (b) above are fused by an amide linkage via a peptide linker or by a direct amide linkage. Such a single-chain antibody is useful in that it can easily be prepared from a transformant carrying an expression vector that comprises the base sequence encoding the protein (a) above, the base sequence encoding a peptide linker (when obtaining a single-chain antibody amide-bound via a linker), and the base sequence encoding the protein (b) above in frame. The base sequence encoding a peptide linker may be any one, as long as it does not contain stop codon in frame with the base sequences encoding the proteins (a) and (b) above.

A peptide linker can be selected as appropriate by a method known in the art. Specifically, as a peptide linker, there may be used peptides of optionally chosen length consisting of 1 or more amino acid residues; there may be used, for example, peptides consisting of 10 or more amino acid residues.

The present invention also provides a polynucleotide encoding the amino acid sequence of the protein of the present invention. The polynucleotide of the present invention may be any one, as long as it contains the aforementioned base sequence encoding the protein of the present invention.

Specifically, as the polynucleotide of the present invention, there may be mentioned base sequences encoding the protein (a) above (e.g., base sequence shown by SEQ ID NO:1), base sequences encoding the protein (b) above (e.g., base sequence shown by SEQ ID NO:3), and base sequences encoding the single-chain antibody above. As the polynucleotide of the present invention, there may also be mentioned a polynucleotide having the base sequence encoding a protein obtained by gene recombination of the protein of the present invention.

The aforementioned polynucleotide of the present invention can be obtained using a method known per se according to the disclosure herein. For example, the polynucleotide of the present invention can be obtained from a hybridoma that produces an anti-plasticizer monoclonal antibody, which is not to be construed as limitative. The N-terminal amino acid sequence of the antibody protein is determined, a primer having a base sequence deduced from this amino acid sequence is then prepared, mRNA is prepared from an antibody-producing hybridoma by a methods known per se, and single-stranded cDNA is synthesized on the basis of the mRNA using reverse transcriptase, after which the polynucleotide of the present invention can be obtained selectively using PCR method, hybridization method, etc. on the basis of the amino acid sequence or base sequence of the variable region of a heavy chain or light chain of an anti-plasticizer monoclonal antibody disclosed herein. Such methods are well-known; those skilled in the art can easily isolate the polynucleotide of the present invention on the basis of the disclosure herein. As specific procedures for these methods, there may be mentioned, for example, the method described in Molecular Cloning, 3rd edition (J. Sambrook et. al., Cold Spring Harbor Lab. Press, 2001) and the like. Useful methods of mRNA extraction include the method described in the operating manual for the Amersham QuickPrep mRNA purification kit; useful methods of cDNA synthesis and 5′-RACE include the methods described in the instruction manual for the CLONTECH Laboratories SMART RACE kit.

In an embodiment, the complex of the present invention can be a recombinant antibody (including a fragment thereof). Regarding to prepare recombinant antibodies and the like, Chapter 2 of “RECOMBINANT ANTIBODIES” [edited by F. Breitling, John Wiley & Sons (USA), 1999] describes a method of preparing recombinant antibody fragments, a method of cloning antibody genes from hybridoma cell lines, a method of preparing antibody gene libraries, a method of selection of recombinant antibodies from gene libraries, a method of antibody engineering, etc., using which methods recombinant antibodies can be prepared.

Chapter 4 of the same book describes a method of producing recombinant antibodies, and methods of their expression, including expression in rabbit reticulocyte lysate in vitro; expression in prokaryotes such as E. coli cytoplasm, soluble fraction of periplasm, inclusion body of periplasm, Bacillus and Streptomyces; and also described are methods of expression in eukaryotes such as Pichia, Saccharomyces, Schizosaccharomyces and other yeasts, Trichoderma and other fungi; expression in insect cells such as Baculovirus; expression in animal cells such as myeloma, CHO, and COS, transgenic plants such as of tobacco, and transgenic animals, using which methods transformants can be prepared.

Chapter 4 of the same book also describes methods of purifying recombinant antibodies, wherein the desired product is first separated by a physical means, for example, cell harvest by centrifuging transformant, cell disruption by ultrasonication etc., mechanical milling, or enzymatic lysis. Subsequently, purification is conducted using a combination of ion exchange chromatography, size exclusion chromatography, thiophilic adsorption chromatography, affinity chromatography, etc. Affinity chromatography, in particular, is an efficient method; the desired product can be produced by purification using an antigen-specific method based on antigen recognition specificity, an antibody-specific method based on binding of protein A or protein G to the Fc portion or Fab′ portion, or in the case of scFv without these portions, a method comprising expressing the scFv as a fusion antibody having a small peptide fragment called “tag” and an affinity column specific for this tag is used (e.g., His-tag, c-myc tag, Strep tag, etc.).

First, a cDNA library of anti-plasticizer monoclonal antibody-producing cells is constructed, and the cDNA library is then screened using a cDNA encoding the N-terminal sequence of the highly conservative constant region or the variable region of a heavy or light chain of an immunoglobulin as a probe; anti-plasticizer monoclonal antibody light or heavy chain cDNA can be thus isolated. As specific procedures for these methods, there may be mentioned, for example, the method described in Molecular Cloning, 3rd edition (J. Sambrook et. al., Cold Spring Harbor Lab. Press, 2001).

The polynucleotide of the present invention may also be synthesized chemically using a well-known technique on the basis of the sequence described herein.

Methods of gene engineering of the protein of the present invention include methods known per se; there may be used, for example, methods of converting the base sequence encoding the protein. Conversion of base sequences of polynucleotide (e.g., DNA) can be achieved by a method known per se such as the ODA-LAPCR method, the Gupped duplex method, or the Kunkel method, or a method based thereon, using PCR or a known kit such as Mutan™-Super Express Km (Takara Shuzo Co., Ltd.) or Mutan™-K (Takara Shuzo Co., Ltd.) and the like. Depending on the purpose of use, the cloned antibody protein-encoding DNA can be used as is, or after being digested with a restriction enzyme or added with a linker as necessary. This DNA may have ATG as a translation initiation codon on the 5′-terminal side thereof, and may have TAA, TGA or TAG as a translation stop codon on the 3′-terminal side thereof. These translation initiation codons and translation stop codons can also be added using an appropriate synthetic DNA adapter. An expression vector for the antibody protein of the present invention can, for example, be produced by (a) cleaving the desired DNA fragment from the DNA encoding the antibody protein of the present invention, and (b) joining the DNA fragment to downstream of a promoter in an appropriate expression vector.

Methods of Preparing Recombinant Antibodies

Various forms of recombinant antibodies can be prepared and are exemplified by those described in Roland Kontermann's ANTIBODY ENGINEERING HOME PAGE (http://aximt1.imt.uni-marburg.de/ rek/AEP.html, Feb. 25, 2002); for example, recombinant antibodies can be prepared in the form of Fab′ fragments, F(ab′) fragments, Fv fragments (Fv), single-chain Fv fragments (scFv), bispecific-chimeric scFV (χ-scFv), tandem scFV (scFv)2, bispecific-(scFv)2, disulfide-linked scFv, disulfide-stabilized Fv fragments (dsFv), diabody, single-chain diabody (scDb), bivalent diabody, bispecific diabody, knob-into-hole stabilized diabody, disulfide-stabilized diabody, triabody, tetrabody, trispecific triabody, CL-dimerized scFv, CH1-CL-dimerized scFv, CH3-dimerized scFv, knob-into-hole CH3-dimerized scFv, CH3-dimerized bivalent diabody, Fc-dimerized scFv, Fab-scFv fusions, Ig-scFv fusions, leucine-zipper stabilized scFv dimers, helix-stabilized scFv dimers, 4 helix-bunde stabilized scFv tetramers, streptavidin-scFv, and intrabody.

Methods wherein an antibody having a desired useful property is selected by shuffling mutated antibody genes also fall in the scope of the present invention.

Recombinant antibody expression systems Any recombinant antibody expression system can be used, as long as it is capable of efficiently expressing a recombinant antibody; for example, as tabulated in Roland Kontermann's ANTIBODY ENGINEERING HOME PAGE (http://aximt1.imt.uni-marburg.de/ rek/AEP.html, Feb. 25, 2002), expression of Fv, scFv and scFv derivatives, bivalent and bispecific scFv, scFv or Fab-fusion proteins, intrabodies, etc. in mammalian cells, expression of scFV, Fab, etc. in insect cells, expression of Fv, scFv, Fab, etc. in fungal cells, and expression of scFv in plant cells are known, and hence a variety of expression systems can be used.

Methods of Constructing a cDNA Library

Any method of constructing a cDNA library can be used, as long as it is capable of efficiently preparing a cDNA library; such methods include the phage display method described in Roland Kontermann's ANTIBODY ENGINEERING HOME PAGE (http://aximt1.imt.uni-marburg.de/ rek/AEP.html, Feb. 25, 2002).

Selection Methods of Recombinant Antibody

As useful methods of selecting the desired recombinant antibody from the library constructed, the methods described in Roland Kontermann's ANTIBODY ENGINEERING HOME PAGE (http://aximt1.imt.uni-marburg.de/ rek/AEP.html, Feb. 25, 2002), such as the protocol “Isolation of Recombinant Antibodies from Phagemid Libraries” and “Isolation of Peptides from fd Phage Libraries” can be mentioned.

Depending on the purpose of use, the polynucleotide (for example, DNA) can be used as is, or after being cleaved or added with another polynucleotide as necessary. For example, this DNA may have the translation initiation codon ATG on a terminal thereof. These modifications can be achieved by a method known per se, for example, a method described in Molecular Cloning, 3rd edition (J. Sambrook et. al., Cold Spring Harbor Lab. Press, 2001), and the like.

By incorporating the thus-obtained DNA, a promoter, a translation initiation codon, the appropriate signal sequence, etc., by a methods known per se, into a vector, a recombinant vector can be produced. As such vectors, promoters and host strains, there may be mentioned, for example, the vectors, promoters and Escherichia strains described in Appendix 3 to Molecular Cloning, 3rd edition (J. Sambrook et. al., Cold Spring Harbor Lab. Press, 2001).

In addition to the aforementioned vectors, useful vectors include plasmids of Escherichia coli origin (pET-276, pCANTAB-5E, pUC19, pT7Bule T), plasmids of Bacillus subtilis origin (e.g., pUB110, pTP5, pC194), plasmids of yeast origin (e.g., pSH19, pSH15), λ phage, bacteriophages such as M13K07, and animal viruses such as retrovirus, vacciniavirus and vaculovirus, as well as pA1-11, pXT1, pRc/CMV, pRc/RSV, pcDNAI/Neo and the like.

Any promoter can be used, as long as it is suitable for the host used for gene expression. Preferred promoters include, for example, the trp promoter, the lac promoter, the recA promoter, the λP_{L} promoter, and the lpp promoter when the host is a bacterium of the genus Escherichia; the SPO1 promoter, the SPO2 promoter, and the penP promoter when the host is a bacterium of the genus Bacillus; and the PHO5 promoter, the PGK promoter, the GAP promoter, and the ADH promoter when the host is a yeast. When the host is an animal cell, the SRα promoter, the SV40 promoter, the LTR promoter, the CMV promoter, the HSV-TK promoter, etc. are preferred; when the host is an insect cell, the polyhedrin promoter, the P10 promoter, etc. are preferred.

In addition to the aforementioned promoters, the expression vector may contain an enhancer, splicing signal, polyA addition signal, selection marker, SV40 replication origin, etc. as necessary. Selection markers include, for example, the ampicillin resistance gene (hereinafter also referred to as Amp^{R}), the kanamycin resistance gene (hereinafter also referred to as Km^{R}), and the chloramphenicol resistance gene (hereinafter also referred to as Cm^{R}).

Where necessary, a signal sequence suitable for the host is added to the N-terminus side of the antibody protein of the present invention. Useful signal sequences include the phoA signal sequence, the ompA signal sequence and the like when the host is a bacterium of the genus Escherichia; the α-amylase signal sequence, the subtilisin signal sequence and the like when the host is a bacterium of the genus Bacillus; the MFα signal sequence, the SUC2 signal sequence and the like when the host is a yeast; and the insulin signal sequence, the α-interferon signal sequence, and the antibody molecule signal sequence and the like when the host is an animal cell. Using the thus-constructed vector harboring the DNA encoding the antibody protein of the present invention, a transformant can be produced.

Useful hosts include the genus Escherichia, the genus Bacillus, yeasts, insect cells, insects, animal cells and the like. Examples of useful bacteria of the genus Escherichia include Escherichia coli K12-DH1 [Proc. Natl. Acad. Sci. USA, vol. 60, 160 (1968)], JM103 [Nucleic Acids Research, vol. 9, 309 (1981)], JA221 [Journal of Molecular Biology, vol. 120, 517 (1978)], HB101 [Journal of Molecular Biology, vol. 41, 459 (1969)], C600 [Genetics, vol. 39, 440 (1954)], BL21DE3(pLysS), TG-1, and JM109. Useful bacteria of the genus Bacillus include, for example, Bacillus subtilis MI114 [Gene, vol. 24, 255 (1983)] and 207-21 [Journal of Biochemistry, vol. 95, 87 (1984)]. Useful yeasts include, for example, Saccharomyces cerevisiae AH22, AH22R⁻, NA87-11A, DKD-5D, and 20B-12, Schizosaccharomyces pombe NCYC1913 and NCYC2036, and Pichia pastoris. When the virus is AcNPV, useful insect cells include, for example, established Spodoptera frugiperda larva cells (Sf cells), Trichoplusia ni midgut MG1 cells, Trichoplusia ni egg High Five™ cells, Mamestrabrassicae cells, Estigmena acrea cells and the like. When the virus is BmNPV, silkworm-derived established cells (Bombyx mori N; BmN cells) etc. are used. Such Sf cells include, for example, Sf9 cells (ATCC CRL1711) and Sf21 cells [both described by Vaughn, J. L. et al. In Vivo, 13, 213-217 (1977)]. Useful insects include, for example, silkworm larvae [Maeda et al., Nature, vol. 315, 592 (1985)]. Useful animal cells include, for example, simian COS-7 cells, Vero, Chinese hamster CHO cells, mouse L cells, mouse AtT-20, mouse myeloma cells, rat GH3 and human FL cells.

Transformation of bacteria of the genus Escherichia can, for example, be achieved according to the methods described in Proc. Natl. Acad. Sci. USA, vol. 69, 2110 (1972) and Gene, vol. 20 17, 107 (1982). Transformation of bacteria of the genus Bacillus can, for example, be achieved according to the method described in Molecular & General Genetics, vol. 168, 111 (1979). Transformation of yeasts can, for example, be achieved according to the methods described in Methods in Enzymology, vol. 194, 182-187 (1991) and Proc. Natl. Acad. Sci. USA, vol. 75, 1929 (1978). Transformation of insect cells or insects can, for example, be achieved according to the method described in Bio/Technology, vol. 6, 47-55 (1988). Transformation of an animal cell can, for example, be achieved using the method described in SAIBO KOGAKU (CELL TECHNOLOGY), Supplementary 8: Shin Saibou Kougaku Jikken Protocol, 263-267 (1995) (published by Shujunsha Co., Ltd.) and Virology, Vol. 52, 456 (1973). Thus, a transformant transformed with an expression vector harboring the polynucleotide encoding an antibody protein is obtained.

Furthermore, the protein of the present invention can be produced by cultivating the thus-obtained transformant and harvesting the resulting protein.

Regarding culture media, liquid media are suitable for the cultivation of a transformant whose host is a bacterium of the genus Escherichia or Bacillus, which media are supplemented with carbon sources, nitrogen sources, minerals, and other substances required for the growth of the transformant. Carbon sources include, for example, glucose, dextrin, soluble starch, sucrose and the like; nitrogen sources include, for example, inorganic or organic substances such as ammonium salts, nitrates, corn steep liquid, peptone, casein, meat extract, soybean flour, potato extract and the like; minerals include, for example, calcium chloride, sodium dihydrogen phosphate, magnesium chloride and the like. Yeast, vitamins, growth promoters, etc. may also be added. It is desirable that the pH of the medium be about 5 to 8.

Preferred media for cultivating the genus Escherichia include, for example, M9 medium supplemented with glucose and casamino acid [Miller, Journal of Experiments in Molecular Genetics, 431-433, Cold Spring Harbor Laboratory, New York (1972)). To increase promoter efficiency where necessary, an agent like 3β-indolylacrylic acid or isopropylthiogalactoside (IPTG), for example, may be added. When the host is a bacterium of the genus Escherichia, cultivation is normally carried out at about 15 to 43° C. for about 3 to 24 hours, and aeration and stirring may be conducted as necessary. When the host is a bacterium of the genus Bacillus, cultivation is normally carried out at about 30 to 40° C. for about 6 to 24 hours, and aeration and stirring may be conducted as necessary. Media for cultivating a transformant whose host is a yeast include, for example, Burkholder's minimum medium [Bostian, K. L. et al., Proc. Natl. Acad. Sci. USA, vol. 77, 4505 (1980)] and SD medium supplemented with 0.5% casamino acid [Bitter, G. A. et al., Proc. Natl. Acad. Sci. USA, vol. 81, 5330 (1984)]. It is preferable that the medium's pH be adjusted to about 5 to 8. Cultivation is normally carried out at about 20 to 35° C. for about 24 to 72 hours, with aeration and stirring conducted as necessary.

Useful media for cultivating a transformant whose host is an insect cell or an insect include Grace's insect medium [Grace, T. C. C., Nature, 195, 788 (1962)] supplemented with additives such as inactivated 10% bovine serum as appropriate. It is preferable that the medium's pH be adjusted to about 6.2 to 6.4. Cultivation is normally carried out at about 27° C. for about 3 to 5 days, with aeration and stirring conducted as necessary. Useful media for cultivating a transformant whose host is an animal cell include, for example, MEM medium supplemented with about 5 to 20% fetal bovine serum [Science, vol. 122, 501 (1952)], DMEM medium [Virology, vol. 8, 396 (1959)], RPMI1640 medium [The Journal of the American Medical Association], vol. 199, 519 (1967)], and 199 medium [Proceeding of the Society for the Biological Medicine, vol. 73, 1 (1950)]. It is preferable that pH be about 6 to 8. Cultivation is normally carried out at about 30 to 40° C. for about 15 to 60 hours, with aeration and stirring conducted as necessary. As described above, the antibody protein of the present invention can be produced in the cells or cell membrane of the transformant or outside the cells.

Separation and purification of the desired antibody protein of the present invention from the thus-obtained culture can, for example, be achieved by the method described below. When extracting the antibody protein of the present invention from cultured cells, there may be used as appropriate, for example, a method wherein cultured cells are collected by a known means, suspended in an appropriate buffer solution, and disrupted by means of ultrasound, lysozyme and/or freeze-thawing etc., after which a crude extract of antibody protein is obtained by centrifugation or filtration. The buffer solution may contain a protein denaturant such as urea or guanidine hydrochloride and a surfactant such as Triton X-100™. If the antibody protein is secreted in the culture broth, it is treated by a method known per se to separate the cells and the supernatant after completion of cultivation and the supernatant is collected. Purification of the antibody protein contained in the thus-obtained culture supernatant or extract can be achieved by appropriately combining methods known per se of separation and purification. Useful known methods of separation and purification include methods based on solubility, such as salting-out and solvent precipitation; methods based mainly on molecular weight differences, such as dialysis, ultrafiltration, gel filtration, and SDS-polyacrylamide gel electrophoresis; methods based on charge differences, such as ion exchange chromatography; methods based on specific affinity, such as affinity chromatography; methods based on hydrophobicity differences, such as reversed-phase high performance liquid chromatography; and methods based on isoelectric point differences, such as isoelectric focusing.

The complex, protein and partial peptide of the present invention and/or a salt thereof can be produced by a method known per se of protein synthesis, or by cleaving the protein of the present invention using an appropriate protease. This protein synthesis can, for example, be achieved by solid phase synthesis or liquid phase synthesis. Specifically, the desired protein can be produced by condensing the partial peptide or amino acids that constitute the protein of the present invention and the remainder, and if the purified product has a protective group, removing the protective group. Known methods of condensation and the methods of protective group removal described below, for example, can be used.

(1) M. Bodanszky and M. A. Ondetti, Peptide Synthesis, Interscience Publishers, New York (1966)

(2) Schroeder and Luebke, The peptide, Academic Press, New York (1965)

(3) Nobuo Izumiya et al., Peputido Gosei no Kiso to Jikken, Maruzen Co., Ltd. (1975)

(4) Haruaki Yajima and Shunpei Sakakibara, Seikagaku Jikken Kouza 1, Tanpakushitsu no Kagaku IV, 205, (1977)

(5) Haruaki Yajima, supervisor, Zoku lyakuhin no Kaihatsu, Vol. 14: Peputido Gosei, Hirokawa Publishing

After the reaction, the protein of the present invention can be purified and isolated using ordinary purification methods such as solvent extraction, distillation, column chromatography, liquid chromatography, and recrystallization in combination. If the protein obtained by the method described above is a free from, it can be converted to an appropriate salt by a known method; if the protein obtained is a salt, it can be converted to a free form by a known method.

The thus-obtained complex and/or protein of the present invention can be used as a reagent for quantitatively measuring a plasticizer, or can be used to produce affinity columns for concentrating a plasticizer, in which it is immobilized to various carriers. Additionally, by identifying a plasticizer that binds (i.e., cross-reacts) to the complex and/or protein of the present invention, the applicability of the complex and/or protein of the present invention can be expanded. Furthermore, the present invention provides a kit for measuring or quantifying a plasticizer and a kit for concentrating a plasticizer, each of which containing the complex and/or protein of the present invention.

Although the aforementioned kit may contain only one kind of the complex and/or protein of the present invention, it may also contain plural kinds of complex and/or protein of the present invention. For example, by using a kit containing a plurality of complexes of different degrees of cross-reactivity, a particular plasticizer can be determined or quantified with specificity.

As methods of measuring a plasticizer using the complex and/or protein of the present invention, there may be mentioned various methods in common use for antigen detection, such as radioisotope immunoassay (RIA), ELISA [Engvall, E., Methods in Enzymol., 70, 419-439 (1980)], fluorescent antibody method, plaque method, spot method, agglutination method, and Ouchterlony method (“Hybridoma Method and Monoclonal Antibodies”, published by R&D Planning, pages 30-53, Mar. 5, 1982). From the viewpoint of sensitivity, simplicity, etc., ELISA is commonly used.

As a carrier for the complex and/or protein of the present invention, there may be mentioned, for example, microplates (e.g., 96-well microplate, 24-well microplate, 192-well microplate, 384-well microplate, etc.), test tubes (e.g., glass test tubes, plastic test tubes), glass particles, polystyrene particles, modified polystyrene particles, polyvinyl particles, latexes (e.g., polystyrene latex), nitrocellulose membranes, cyanogen bromide-activated filter paper, DBM-activated filter paper, granular solid phases (e.g., Sepharose, Sephadex, agarose, cellulose, Sephacryl, etc.), iron-containing polycarbonate films, and magnet-containing beads.

The complex and/or protein of the present invention can be carried on carriers by methods known per se [e.g., “Enzyme Immunoassay” above, pp. 268-296, “Affinity Chromatography Handbook”, Amersham-Pharmacia Biotech K. K., published Dec. 20, 1998].

In the immunological concentration method of the present invention, the desired substance of minimal immunological impurity contents can be concentrated at rates as high as several thousands to several tens of thousands of times, by passing a large amount of sample through a column of immunological adsorbent or mixing with immunological adsorbent particles to adsorb the desired environmental hormone, degradation products thereof or a mixture thereof to the immunological adsorbent by an antigen-antibody reaction, and subsequently eluting the desired product by known methods, such as changing the pH (lowering to pH 2.5˜3, raising to pH 11.5), changing the ionic strength (1M NaCl etc.), changing the polarity [10% dioxane, 50% ethylene glycol, 3M chaotropic salts (SCN⁻, CCl₃COO⁻, I⁻), etc.], adding protein denaturants (8M urea, 6M guanidine hydrochloride, etc.), and conducting electrophoretic dissociation.

Environmental hormones occurring in very trace amounts in the environment, degradation products thereof, or a mixture thereof can thereby be concentrated at much higher rates than by conventional methods of concentration such as solvent extraction and solid phase extraction and in addition concentrates of lower contents of impurities and other substances that interfere with quantitation can be obtained.

When bases, amino acids and the like are shown in abbreviations in the present specification and Figures, they are based on the abbreviations by IUPAC-IUB Commission on Biochemical Nomenclature or abbreviations conventionally used in the pertinent field. Examples thereof are given in the following. When an amino acid can have an optical isomer, it is an L form, unless particularly indicated.
DNA: deoxyribonucleic acid
cDNA: complementary deoxyribonucleic acid
a,A: adenine
t,T: thymine
g,G: guanine
c,C: cytosine
RNA: ribonucleic acid
mRNA: messenger ribonucleic acid
Abbreviations of amino acids
letters: 1 letter: Japanese name
Gly: G: glycine
Ala: A: alanine
Val: V: valine
Leu: L: leucine
Ile: I: isoleucine
Ser: S: serine
Thr: T: threonine
Cys: C: cysteine
Met: M: methionine
Glu: E: glutamic acid
Asp: D: aspartic acid
Lys: K: lysine
Arg: R: arginine
His: H: histidine
Phe: F: phenylalanine
Tyr: Y: tyrosine
Trp: W: tryptophan
Pro: P: proline
Asn: N: asparagine
Gln: Q: glutamine
Asx: B: Asn+Asp
Glx: Z: Gln+Glu

### EXAMPLES

The present invention is explained in more detail by illustrating Examples in the following, which are not to be construed as limitative.

[Materials]

A Hybridoma that Produces an Anti-DEHP Antibody (DH-150)

A hybridoma line that produces an anti-DEHP antibody (DH-150) (isotype γ2a, K), DH-150, was prepared per the procedures published by Goda Y. et al. in “Development of the ELISAs for Detection of Endocrine Disrupters”, Fifth International Symposium on Environmental Biotechnology (ISEB2000), Program/Abstracts, p. 119 (2000). This cell line was subcultured using an RPMI1640 medium containing 10% fetal calf serum (medium for hybridoma) (see N. Kobayashi et al., J. Steroid Biochem. Mol. Biol., 64, 171-177 (1998)).

A Hybridoma that Produces an Anti-DEHP Antibody (DF-34)

A hybridoma line that produces an anti-DEHP antibody (DF-34), DF-34 (FERM BP-6635), is described in the pamphlet for International Patent Publication No. WO99/43799. This cell line was subcultured using an RPMI1640 medium containing 10% fetal calf serum (medium for hybridoma).

Primers

For the primers used for synthesis of cDNA and PCR, chemical synthesis and cartridge purification were outsourced to Kurabo Industries, Ltd. or ESPEC OLIGO SERVICE Corporation. The base sequences of the individual primers are shown in Table 1.

TABLE 1
Primers Used in the Examples

| | |
|---|---|
| Name of primer | Base sequence |

| | | |
|---|---|---|
| G2a-CH-1 | 5′ GCTTGCCGGGTGGGCCAC 3′ | (SEQ ID NO:10) |
| G2a-GH-2 | 5′ ACACTGCTGGACAGGGAT 3′ | (SEQ ID NO:11) |
| G2a-CH-3-XmaI | 5′ GGATCCCGGGAGTACCCCTTGACCAGGC 3′ | (SEQ ID NO:12) |
| K-CH-1 | 5′ GTTGAAGCTCTTGACAAT 3′ | (SEQ ID NO:13) |
| K-CH-3-XmaI | 5′ GGATCCCGGGTGGATGGTGGGAAGATG 3′ | (SEQ ID NO:14) |
| AAP | 5′ GGCCACGCGTCGACTAGTACGGGIIGGGIIGGGIIG 3′ | (SEQ ID NO:15) |
| AUAP | 5′ GGCCACGCGTCGACTAGTAC 3′ | (SEQ ID NO:16) |
| MKV-9 | 5′ ACTAGTCGACATGGTRTCCWCASCTCAGTTCCTTG 3′ | (SEQ ID NO:17) |
| KS-back | 5′ GGAAACAGCTATGACCATG 3′ | (SEQ ID NO:18) |
| KS-for | 5′ GTAAAACGACGGCCAGT 3′ | (SEQ ID NO:19) |
| DH-150-VH-5 | 5′ ATTGTTATTACTCGCGGCCCAACCGGCCATGGCCGAGG | (SEQ ID NO:20) |
| | TGCATGGTGGAGTCTGGG 3′ | |
| DH-150-VH-3 | 5′ CCGCCGGATCCACCTCCGCCTGAACCGCCTCCACCTGA | (SEQ ID NO:21) |
| | GGAGACGATGACTGACGTTCC 3′ | |
| DH-150-VL-5 | 5′ CAGGCGGAGGTGGATCCGGCGGTGGCGGATCGGATATC | (SEQ ID NO:22) |
| | CAGATAACACAGATTACA 3′ | |
| DH-150-VL-3 | 5′ GCTCAACTTTCTTGTCGACTTATCATCATCATCTTTAT | (SEQ ID NO:23) |
| | AATCTTTCAGCTCCAGCGTGGTCCCTGC 3′ | |
| G1-CH-1 | 5′ GCTGGCCGGGTGGGCAAC 3′ | (SEQ ID NO:28) |
| MKV-5 | 5′ ACTAGTCGACATGGATTTWCAGGTGCAGATTWTCAGCT | (SEQ ID NO:29) |
| | TC 3′ | |
| DF-34-VH-5 | 5′ ATTGTTATTACTCGCGGCCCAACCGGCCATGGCCGATG | (SEQ ID NO:30) |
| | TACAACTTCAGGAGTCAGGACC 3′ | |
| DF-34-VH-3 | 5′ CCGCCGGATCACCTCCGCCTGAACCGCCTCCACCTGAG | (SEQ ID NO:31) |
| | GAGACGGTGACTGAGGTTCCCT 3′ | |
| DP-34-VL-5 | 5′ CAGGCGGAGGTGGATCCGGCGGTGGCGGATCGCAGATT | (SEQ ID NO:32) |
| | GTTCTCACCCAGTCTCC 3′ | |
| DF-34-VL-3 | 5′ GCTCAACTTTCTTGTCGACTTTATCATCATCATCTTTA | (SEQ ID NO:33) |
| | TAATCTTTTATTTCCAACTTTGTCCCCG 3′ | |

### Example 1

### Cloning of Anti-DEHP Antibody (DH-150) V_{H} Gene

Total RNA was extracted from the hybridoma line DH-150 (1×10⁷ cells) using an RNeasy mini kit (QIAGEN). A primer specific for the γ2a chain (G2a-CH-1) or a primer specific for the κ chain (K-CH-1) and Superscript II reverse transcriptase (Invitrogen) (1 μL) were added to this RNA (4.2 μg), and the RNA was incubated in the attached buffer solution (25 μL) at 42° C. for 50 minutes. After incubation at 70° C. for 15 minutes to inactivate the enzyme, the crude reaction liquid was purified using a GlassMAX spin cartridge (Invitrogen) to yield first strand cDNAs containing the V_{H} or V_{L} gene (V_{H}-cDNA and V_{L}-cDNA), respectively. Subsequently, 5′-RACE using V_{H}-cDNA as the template [5′RACE system for rapid amplification of cDNA ends, version 2.0 (Invitrogen)] was conducted to obtain a gene fragment of the V_{H} domain. Specifically, deoxycytosine triphosphate (dCTP) (5 nmol) and terminal deoxynucleotidyl transferase (TdT) (1 μL) were added to a cDNA solution (10 μL), and the reaction was carried out in TdT buffer solution (25 μL) at 37° C. for 10 minutes. Subsequently, PCR [95° C., 1 minute; 64° C., 1 minute; 72° C., 2 minutes (35 cycles), then 72° C., 10 minutes] was conducted in Ex-Taq buffer solution (40 μL) using primers complementary to the poly-C sequence and the γ2a chain constant region (AAP and G2a-CH-2, respectively) (20 pmol each) and Ex-Taq DNA polymerase (Takara Shuzo) (1 U). Furthermore, nested PCR (liquid volume 100 μL) using the primers AUAP and G2a-CH-3-XmaI (50 pmol each) and Ex-Taq DNA polymerase (2.5 U) with a 1000 fold diluted solutions of this PCR reaction liquid (10 μL) as the template was conducted under the same reaction conditions as above. The resulting crude reaction liquid was subjected to electrophoresis (TAE buffer solution; 50 V) using low-melting agarose (SeaPlaque; BMA) (2%), and an about 800-bp band was recovered using a QIAquick gel extraction kit (Qiagen) to obtain the desired DNA fragment containing the V_{H} gene (V_{H}-DNA).

### Example 2

### Cloning of Anti-DEHP Antibody (DH-150) V_{L} Gene

A PCR with the above-described V_{L}-cDNA (10 μL of 1000 fold diluted solution) as the template was attempted using a combination of one of the previously reported primers MKV-1 to 1 for cloning the mouse variable region gene (see S. T. Jones et al., Biotechnology, 9, 88-89 (1991)) and K—CH-3-XmaI (50 pmol each). For this PCR [95° C., 1 minute; 50° C., 1 minute; 72° C., 3 minutes (35 cycles), then 72° C., 10 minutes], the reaction was carried out in Pfu buffer solution (100 μL) using Pfu DNA polymerase (Promega) (3 U). A portion of the crude reaction liquid was subjected to agarose electrophoresis; a band of a size (about 400 bp) expected with the use of the MKV-9 primer was clearly observed. Hence, the remaining portion of the reaction liquid was purified by the above-described method to obtain the desired DNA fragment containing the V_{L} gene (V_{L}-DNA).

### Example 3

### Subcloning of Anti-DEHP Antibody (DH-150) V_{H} and V_{L} Gene

Xma I (40 U) was added to each of the above-described V_{H}-DNA and V_{L}-DNA (calculated value 1.5 μg each), and these were incubated at 37° C. overnight. After the reaction liquid was extracted with phenol/chloroform/isoamyl alcohol (PCI), ethanol precipitation was conducted; SalI (40 U) was added to the resulting precipitate, and the precipitate was again incubated at 37° C. overnight. The reaction liquid was subjected to PCI extraction/ethanol precipitation, and then subjected to electrophoresis using low-melting agarose as described above to purify the desired gene fragment. These DNAs (0.1 μg) were mixed with the pBluescript II vector (0.25 μg), previously treated with XmaI/SalI, T4 DNA ligase (New England Biolabs) (1600 U) was added, and these were incubated at 16° C. overnight. The reaction liquid was subjected to PCI extraction/ethanol precipitation to purify, and the resulting recombinant plasmid was transformed into XL1-Blue Subcloning-grade competent cells (Stratagene) by the heat shock method. The transformation liquid was applied to an ampicillin-containing 2×YT-agar plate, and incubated at 37° C. overnight. Some of the resulting transformant clones (4 clones for each of V_{H}-DNA and V_{L}-DNA) were optionally selected, and cultured in an ampicillin-containing 2×YT medium (10 mL); after being prepared in a 15% glycerol mixture, each clone was stored at −80° C.

### Example 4

### Determination of the Base Sequences of Anti-DEHP Antibody (DH-150) V_{H} and V_{L} Gene

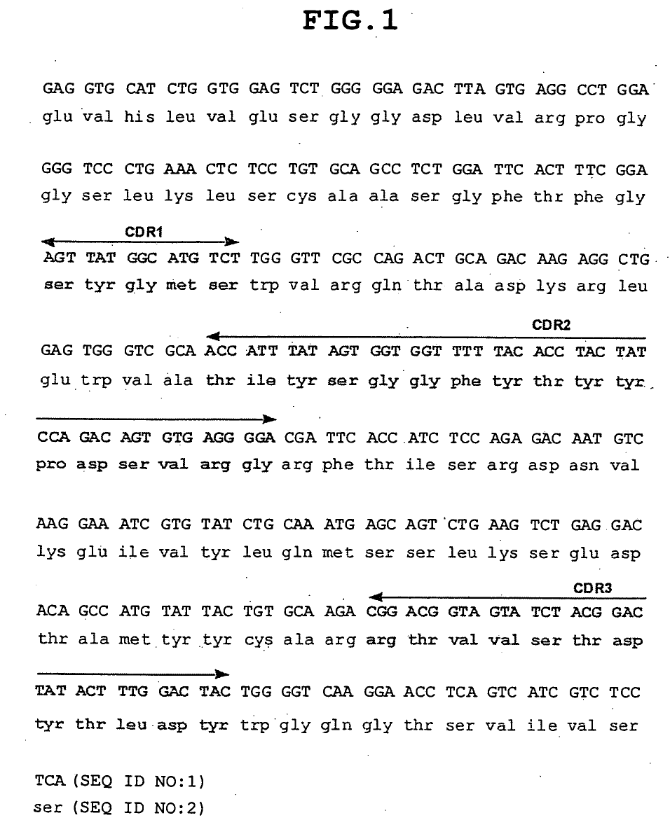

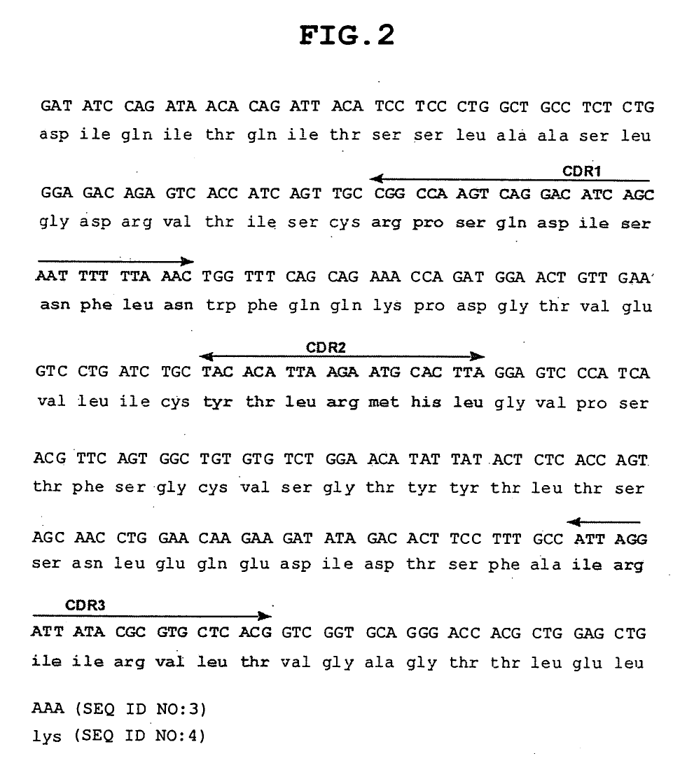

The above-described transformant clones were cultured in an ampicillin-containing 2×YT medium (10 mL), and plasmids were extracted using a QIAGEN plasmid mini kit (Qiagen). A sequencing primer (KS-back or KS-for; 1.8 pmol each) was added to a portion of each plasmid (0.5 or 1.0 μg), and a PCR reaction was carried out using a Dual CyDye terminator sequencing kit (Amersham Biosciences). In this PCR, a cycle of “95° C. for 20 seconds; 55° C. for 15 seconds; and 70° C. for 60 seconds” was repeated 35 times. The reaction liquid was subjected to ethanol precipitation, the resulting amplified DNA was recovered and dissolved in the formamide loading dye (4 μL) attached to the kit, and electrophoresis (6% polyacrylamide gel; TBE buffer solution; 1500 V; 200 minutes) was conducted using a Long-Read Tower DNA sequencer (Amersham Biosciences). From the base sequence data obtained, a consensus sequence among 4 clones was obtained for each of V_{H}-DNA and V_{L}-DNA. The thus-obtained base sequences and deduced amino acid sequences are shown in FIGS. 1 and 2 (V_{H} and V_{L}, respectively). From this result, the subgroups of V_{H} and V_{L} were determined to be III(D) and V, respectively, according to Kabat's classification (see “Sequences of Proteins of Immunological Interest, Fifth Edition”, U.S. Department of Health and Human Service, 1991). Also, by a comparison with Kabat's database (see “Sequences of Proteins of Immunological Interest, Fifth Edition”, U.S. Department of Health and Human Service, 1999), the complementarity-determining regions (CDRs) (amino acid sequences that directly interacts with the antigen to play an important role in the expression of affinity and specificity) in V_{H} and V_{L} were identified (FIGS. 1 and 2).

### Example 5

### Construction of Anti-DEHP Antibody (DH-150) scFv Gene

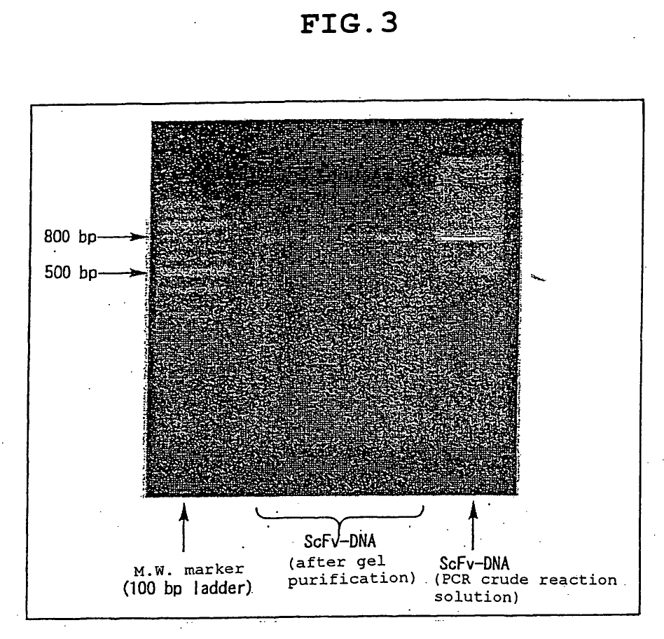

On the basis of the above-described results of gene base sequences, primers specific for the 5′ and 3′ ends of the V_{H} and V_{L} genes (DH-150-VH-5, DH-150-VH-3, DH-150-VL-5, DH-150-VL-3) (Table 1), respectively, were designed, and PCR was conducted with the first strand cDNAs obtained in Example 1 as the templates. An NcoI recognition sequence was introduced to the DH-150-VH-5 primer, and an SalI recognition sequence and the FLAG sequence were introduced to the DH-150-VH-3 primer. Also, a base sequence that encodes the linker sequence (Gly₄Ser)₃ (SEQ ID NO:5) to link V_{H} and V_{L} was added to both the primers DH-150-VH-3 and DH-150-VL-5. The DH-150-VH-5 and DH-150-VH-3 primers (V_{H} amplification) or the DH-150-VL-5 and DH-150-VL-3 primers (V_{L} amplification) (30 pmol each) and Ex-Taq DNA polymerase (2.5 U) were added to a 1:1000 dilution (1 μL) of the above-described cDNA solution, and PCR [95° C., 1 minute; 50° C., 1 minute; 72° C., 3 minutes (35 cycles), then 72° C., 10 minutes] was conducted in Ex-Taq buffer solution (100 μL). The resulting crude reaction liquid was subjected to the above-described electrophoresis using low-melting agarose, and about 400-bp band was recovered using a Wizard PCR preps DNA purification system (Promega) to obtain the desired fragments of the V_{H} gene and the V_{L} gene. Subsequently, these (200 ng each) were mixed, Ex-Taq DNA polymerase (0.65 U) was added, and overlap extension PCR [95° C., 1 minute; 55° C., 1 minute; 72° C., 3 minutes (10 cycles), then 72° C., 10 minutes] was conducted in Ex-Taq buffer solution (25 μL) to construct the scFv gene. Furthermore, the DH-150-VH-5 and DH-150-VL-3 primers (100 pmol each) and Ex-Taq DNA polymerase (2.5 U) were added to a portion (5 μL) of this reaction liquid, and PCR of 25 cycles was conducted under the same conditions (but the reaction liquid volume was 100 μL) to amplify the scFv gene. The resulting crude reaction liquid was subjected to electrophoresis with low-melting agarose, and an about 800-bp band was recovered to obtain the desired scFv gene having the 5′V_{H}-linker-V_{L} 3′ sequence (FIG. 3).

### Example 6

### Cloning, Subcloning, and Base Sequencing of Anti-DEHP Antibody (DF-34) V_{H} and V_{L} Gene

Total RNA was extracted from the hybridoma line DF-34 (1×10⁷ cells) using a RNeasy mini kit (QIAGEN). A primer specific for the γ1 chain (G1-CH-1) or a primer specific for the κ chain (K—CH-1) and Superscript II reverse transcriptase (Invitrogen) (1 μL) were added to this RNA (4 μg), and these were incubated in the attached buffer solution (25 μL) at 42° C. for 50 minutes. After incubation at 70° C. for 15 minutes to inactivate the enzyme, the crude reaction liquid was purified using a GlassMAX spin cartridge (Invitrogen) to obtain first strand cDNAs containing the V_{H} or V_{L} gene, respectively (V_{H}-cDNA and V_{L}-cDNA). Subsequently, 5′-RACE using V_{H}-cDNA as the template was conducted according to the procedures of Example 1 to obtain the desired DNA fragment containing the V_{H} gene (V_{H}-DNA). Separately, a PCR with the above-described V_{L}-cDNA as the template was attempted using a combination of one of 11 primers (MKV-1 to 11) (see S. T. Jones et al., Biotechnology, 9, 88-89 (1991)) and K—CH-3-XmaI (50 pmol each) according to Example 2. A portion of the crude reaction liquid was subjected to agarose electrophoresis; a band of a size (about 400 bp) expected with the use of the primer MKV-5 was clearly observed. Hence, the remaining portion of the reaction liquid was purified by the above-described method to obtain the desired DNA fragment containing the V_{L} gene (V_{L}-DNA).

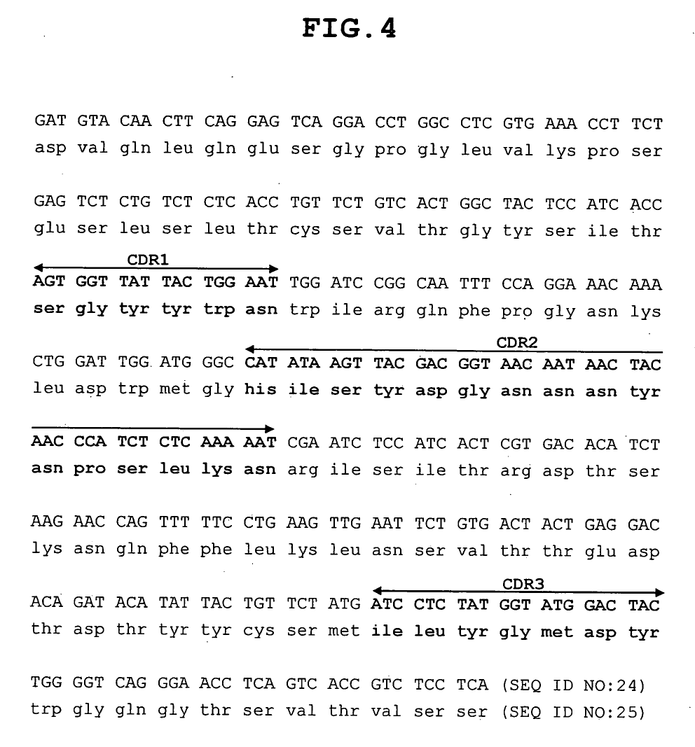

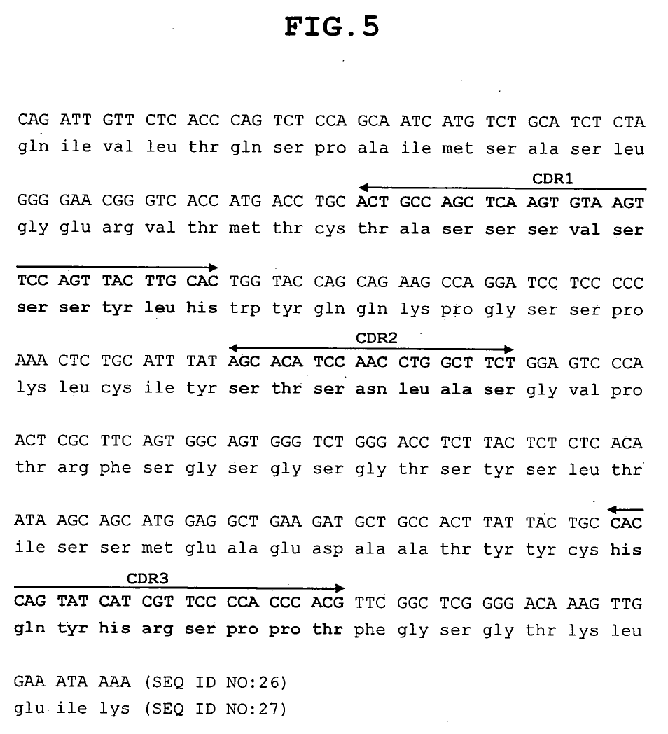

These V_{H}-DNA and V_{L}-DNA (1.5 μg each) were subcloned into the pBluescript II vector according to Example 3 to obtain transformant clones. These clones were cultured in an ampicillin-containing 2×YT medium (10 mL), and plasmids were extracted using a QIAGEN plasmid mini kit (Qiagen). The base sequences of V_{H}-DNA and V_{L}-DNA were determined using a portion of each plasmid (0.5 or 1.0 μg) according to Example 4, and the amino acid sequences were deduced. The results are shown in FIGS. 4 and 5 (V_{H} and V_{L}, respectively). From these results, the amino acid sequence of CDR was determined, and the subgroups of V_{H} and V_{L} were determined to be V_{H}=I (A), V_{L}=IV, respectively.

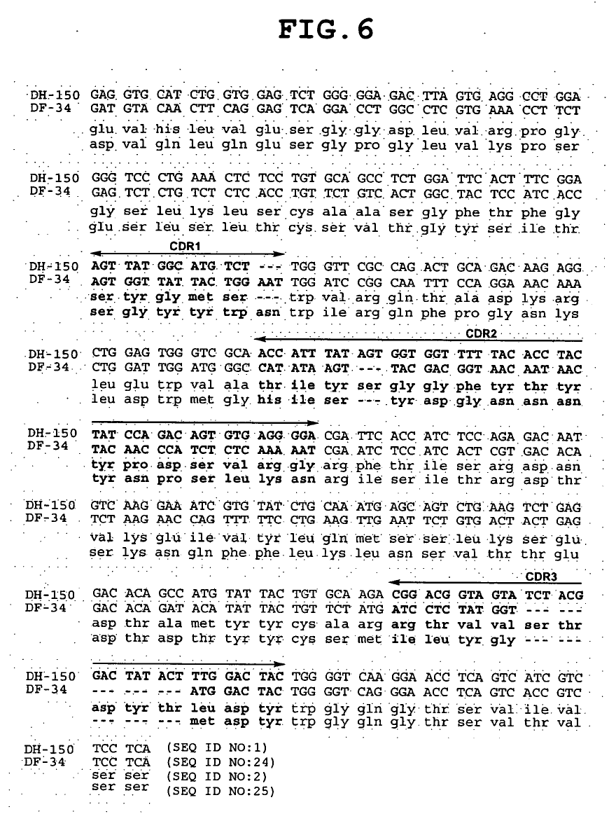

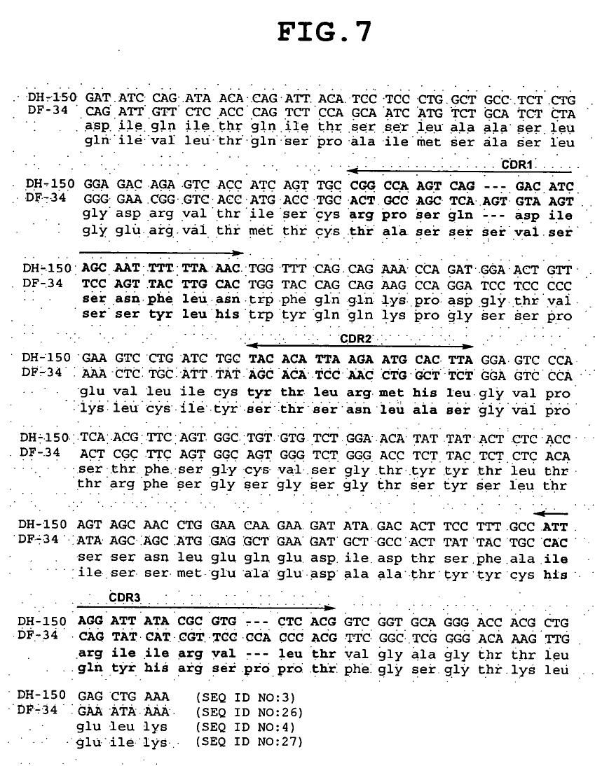

When the sequence data for the DF-34 antibody and the DH-150 antibody were compared, it was found that the homology between the two antibodies was low, as shown in FIGS. 6 and 7 (V_{H} and V_{L}, respectively).

### Example 7

### Construction of Anti-DEHP Antibody (DF-34) scFv Gene

On the basis of the above-described results of gene base sequencing, primers specific for the 5′ and 3′ ends of the V_{H} and V_{L} genes (DF-34-VH-5, DF-34-VH-3, DF-34-VL-5, DF-34-VL-3) (Table 1) were designed, and PCR was conducted with the first strand cDNAs obtained in Example 6 as the templates, as in Example 5. An NcoI recognition sequence was introduced to the DF-34-VH-5 primer, and an SalI recognition sequence and the FLAG sequence were introduced to the DF-34-VL-3 primer. Also, a base sequence that encodes the linker sequence (Gly₄Ser) ₃ to link V_{H} and V_{L} was added to the both primers DF-34-VH-3 and DF-34-VL-5. The resulting fragments of the V_{H} and V_{L} genes (200 ng each) were subjected to overlap extension PCR, the crude reaction liquid was subjected to electrophoresis with low-melting agarose, and an about 800-bp band was recovered to obtain the desired fragment of the scFv gene.

### INDUSTRIAL APPLICABILITY

The present invention has elucidated the amino acid sequences and base sequences of the genes encoding the heavy chain variable region and light chain variable region of an anti-plasticizer antibody. The present invention makes it possible to genetically modify the genes encoding the heavy chain variable region and light chain variable region derived from an anti-plasticizer antibody. For example, by expressing the modified gene in host cells, it has become possible to obtain proteins capable of binding to plasticizers in large amounts, having preferred properties for measuring, quantifying, or concentrating a plasticizer. Also, by using a transformant microorganism and the like having this modified antibody gene, it has also become possible to efficiently produce a recombinant protein. Furthermore, by introducing random mutations in the base sequences encoding the heavy chain variable region and the light chain variable region to construct a library of mutant scFvs, and selecting a mutant showing higher affinity for plasticizers than the original antibody from this library, it has become possible to obtain a recombinant protein having improved affinity for plasticizers. Thus, it has become possible to prepare enzyme immunoassay kits and antibody affinity columns of excellent performance at decreased costs.

This application is based on a patent application No. 2003-110877 filed in Japan on Apr. 15, 2003, the contents of which are hereby incorporated by reference.

1. A protein of the following (a) or (b) or a partial peptide thereof, or a salt thereof:
(a) a protein having the amino acid sequence shown by SEQ ID NO:2, the amino acid sequence shown by SEQ ID NO:25, or an amino acid sequence substantially the same as these;
(b) a protein having the amino acid sequence shown by SEQ ID NO:4, the amino acid sequence shown by SEQ ID NO:27, or an amino acid sequence substantially the same as these.

2. A protein of any of the following (a1) to (a4) and (b1) to (b4) or partial peptide thereof, or a salt thereof:
(a1) a protein having an amino acid sequence having one or two or more amino acids deleted, substituted or added in the amino acid sequence shown by SEQ ID NO:2, which binds to a plasticizer when forming a complex with the amino acid sequence shown by SEQ ID NO:4 or SEQ ID NO:27;
(a2) a protein having an amino acid sequence having one or two or more amino acids deleted, substituted or added in the amino acid sequence shown by SEQ ID NO:2, which binds to a plasticizer when forming a complex with a protein having an amino acid sequence having one or two or more amino acids deleted, substituted or added in the amino acid sequence shown by SEQ ID NO:4 or SEQ ID NO:27;
(a3) a protein having an amino acid sequence having one or two or more amino acids deleted, substituted or added in the amino acid sequence shown by SEQ ID NO:25, which binds to a plasticizer when forming a complex with the amino acid sequence shown by SEQ ID NO:4 or SEQ ID NO:27;
(a4) a protein having an amino acid sequence having one or two or more amino acids deleted, substituted or added in the amino acid sequence shown by SEQ ID NO:25, which binds to a plasticizer when forming a complex with a protein having an amino acid sequence having one or two or more amino acids deleted, substituted or added in the amino acid sequence shown by SEQ ID NO:4 or SEQ ID NO:27;
(b1) a protein having an amino acid sequence having one or two or more amino acids deleted, substituted or added in the amino acid sequence shown by SEQ ID NO:4, which binds to a plasticizer when forming a complex with a protein having the amino acid sequence shown by SEQ ID NO:2 or SEQ ID NO:25;
(b2) a protein having an amino acid sequence having one or two or more amino acids deleted, substituted or added in the amino acid sequence shown by SEQ ID NO:4, which binds to a plasticizer when forming a complex with a protein having an amino acid sequence having one or two or more amino acids deleted, substituted or added in the amino acid sequence shown by SEQ ID NO:2 or SEQ ID NO:25:
(b3) a protein having an amino acid sequence having one or two or more amino acids deleted, substituted or added in the amino acid sequence shown by SEQ ID NO:27, which binds to a plasticizer when forming a complex with a protein having the amino acid sequence shown by SEQ ID NO:2 or SEQ ID NO:25:
(b4) a protein having an amino acid sequence having one or two or more amino acids deleted, substituted or added in the amino acid sequence shown by SEQ ID NO:27, which binds to a plasticizer when forming a complex with a protein having an amino acid sequence having one or two or more amino acids deleted, substituted or added in the amino acid sequence shown by SEQ ID NO:2 or SEQ ID NO:25.

3. A protein of the following (a) or (b) or a partial peptide thereof, or a salt thereof:
(a) a protein having the amino acid sequence shown by SEQ ID NO:2 or an amino acid sequence substantially the same as these;
(b) a protein having the amino acid sequence shown by SEQ ID NO:4 or an amino acid sequence substantially the same as these.

4. The protein of claim 2, wherein the plasticizer is a plasticizer represented by formula (1): 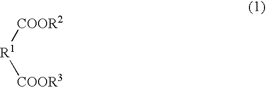
wherein R¹ represents o-phenylene, and R² and R³ are the same or different and each represents H, a linear or branched alkyl having 1 to 20 carbon atoms, a benzyl that may be substituted, or a cyclohexyl that may be substituted.

5. A method of genetically recombining a protein of claim 1.

6. A method of genetically recombining the protein of claim 2.

7. A method of genetically recombining the protein of claim 3.

8. A polynucleotide encoding the protein of claim 1.

9. A polynucleotide encoding the protein of claim 2.

10. A polynucleotide encoding the protein of claim 3.

11. (cancelled)

12. A complex wherein the following (a) and (b) are linked:
(a) a protein having the amino acid sequence shown by SEQ ID NO:2, the amino acid sequence shown by SEQ ID NO:25, or an amino acid sequence substantially the same as these;
(b) a protein having the amino acid sequence shown by SEQ ID NO:4, the amino acid sequence shown by SEQ ID NO:27, or an amino acid sequence substantially the same as these.

13. A method of identifying a plasticizer that binds to the complex of claim 12, which comprises using the complex.

14. A method of measuring or quantifying a plasticizer, which comprises using the complex of claim 12.

15. A kit for measuring or quantifying a plasticizer, which comprises the complex of claim 12.

16. A method of concentrating a plasticizer, which comprises using the complex of claim 12.

17. A kit for concentrating a plasticizer, which comprises the complex of claim 12.
